(19) United States
(12) Patent Application Publication
(10) Pub. No.: US 2006/0105965 A1
(43) Pub. Date: May 18, 2006

(54) Method for enhancing energy levels and reducing the effects of stress using nutraceutical formulations

(75) Inventor: Debasis Bagchi, Concord, CA (US)

Correspondence Address: FLIESLER MEYER, LLP, FOUR EMBARCADERO CENTER, SUITE 400, SAN FRANCISCO, CA, 94111, US

(73) Assignee: InterHealth Nutraceuticals Inc., Benicia, CA (US)

(21) Appl. No.: 11/274,959

(22) Filed: Nov. 15, 2005

Related U.S. Application Data
(60) Provisional application No. 60/628,381, filed on Nov. 16, 2004.

(51) Int. Cl. A61K 31/70; A61K 31/555; A61K 31/522; A61K 31/366; A61K 31/198

(52) U.S. Cl. 514/23; 514/184; 514/263.31; 514/567; 514/553; 514/563; 514/453

(57) ABSTRACT

The present invention provides compositions and methods related to enhancing energy levels in an individual. The invention also provides methods for reducing the effects of stress in an individual. Certain embodiments of this invention include a composition comprising niacin bound chromium, D-ribose, a withanolide and an amine selected from the group consisting of phenylalanine, ceffeine, taurine and gltaumine for enhancing energy levels and/or reducing stress in an individual.

### CLAIM OF PRIORITY

This application claims priority to U.S. Provisional Patent Application Ser. No.: 60/628,381, entitled: Method for Enhancing Energy Levels and Reducing the Effects of Stress Using Nutraceutical Formulations, inventors: Debasis Bagchi and Shil Kothari, filed Nov. 16, 2004. This application is herein incorporated by reference in its entirety.

### FIELD OF THE INVENTION

The present invention relates to methods for enhancing energy levels and reducing the effects of stress in an individual using a nutraceutical formulation comprising niacin bound chromium and other natural components. The invention also contemplates methods for improving cardiovascular health.

### BACKGROUND OF THE INVENTION

The present invention relates generally to compositions and related. Methods for providing various health benefits to humans and more particularly to compositions comprising chromium, ribose and other natural extracts and amines, and methods of use to enhance energy levels and reduce the effects of stress thereby providing various health benefits to individuals.

Anaerobic and aerobic metabolic pathways supply energy for skeletal muscle contraction. Anaerobic energy is supplied by creative phosphate (CP) shuttle and anaerobic glycolysis, whereas the aerobic system utilizes adenosine tri-phosphate (ATP) as the primary energy source for skeletal muscle and energy needed for day-to-day activity. Carbohydrates and fats act as a source of energy providing the necessary elements muscles need to produce ATP. Decreased levels of ATP and CP prevent the adequate supply of energy to cells, thereby hindering efficient performance of routine activities. Energy levels in an individual may be related to many environmental factors such as stress, diet or illness, and decreased energy levels can be correlated with general feelings of lethargy, depressed mood, and other debilitating disorders. Many agents, including caffeine and various sugars, have been used to try to promote the increase of energy in an individual, but with limited success and noticeable side effects.

Both environmental stress and oxidative stress are serious disorders facing individuals. Environmental stress can induce various internal problems such as increased blood pressure [Vanitallie et al., Metabolism (2002) 51 (6 Suppl. 1): 40-5], cardiovascular dysfunction [Fenga et al., Psychopathology (2004) 37:64-8] and decreased immunity [Sephton et al., Brain Behav Immun. (2003) 17:321-8]. Oxidative stress may be induced by any injury to the body, and may also be induced by the introduction of oxygen free radicals into the physiological milieu through environmental factors [Howard et al., Redox Rep. (2002) 7: 308-11]. Oxidative stress contributes to a multitude of disorders, including, but not limited to, tumor progression and cell senescence. Modulation of cellular oxidation using an anti-oxidant or by increasing the output of stress regulating proteins results in decreased oxidative stress, and may enhance the body's ability to boost the immune system to mount a stress response.

While several therapeutic compositions and agents have been proposed to address the above disorders individually, very few therapies can address the needs of all the conditions above and improve this multitude of conditions using a single composition. Thus, there exists a need in the art to provide novel compositions and methods for improving the overall health of individuals, including providing increased energy and reducing the detrimental effects of stress. The present invention fulfills this need and provides further related advantages.

### SUMMARY OF THE INVENTION

The present invention addresses the need in the art to provide compositions and methods to efficiently enhance energy levels and reduce the effects of stress in an individual.

In one aspect, the invention provides a composition comprising chromium, D-ribose, withanolides, and at least one amine. It is contemplated that the withanolides may be derived from Ashwagandha extract or synthesized chemically.

In one embodiment, the amine is selected from the group consisting of phenylalanine, caffeine, taurine and glutamine.

In another embodiment, the invention provides that the chromium comprises niacin-bound chromium (NBC). In a related embodiment, the chromium comprises oxygen-coordinated NBC.

In a further embodiment, the invention provides a composition comprising NBC, D-ribose, withanolides, phenylalanine, caffeine, taurine and glutamine.

In another aspect, the compositions of the invention further comprise a pharmaceutically acceptable carrier or diluent. It is contemplated that the pharmaceutically acceptable carrier or diluent provides the composition in the form of a pill, tablet, capsule, lozenge, gum, liquid, powder, food, beverage or other orally administered form. It is further contemplated that the compositions may be formulated as an injectable solution.

In a further aspect, the invention provides a method for enhancing energy levels in a person or other mammal comprising: administering to the person or other mammal a composition comprising chromium, D-ribose, withanolides, phenylalanine, caffeine, taurine and glutamine in an amount sufficient to enhance energy levels in the person or other mammal.

In a related embodiment, the invention provides a method for reducing stress effects in a person or other mammal comprising: administering to the person or other mammal a composition comprising chromium, D-ribose, withanolides, and at least one amine, in an amount sufficient to reduce stress in the person or other mammal.

In a further related embodiment, the invention contemplates a method for promoting mood elevation in a person or other mammal comprising: administering to the person or other mammal a composition comprising chromium, D-ribose, withanolides, and at least one amine, in an amount sufficient to promote mood elevation in the person or other mammal.

The invention also provides a method for improving cardiovascular health in a person or other mammal comprising: administering to the person or other mammal a composition comprising chromium, D-ribose, withanolides, and at least one amine, in an amount sufficient to promote mood elevation in the person or other mammal.

It is contemplated that when the invention provides a method for promoting mood elevation, a method for reducing stress effects, or a method for improving cardiovascular health, the composition of the method comprises at least one amine selected from the group consisting of phenylalanine, caffeine, taurine and glutamine.

It is further contemplated that in the methods of the invention the chromium comprises NBC. In one embodiment, the chromium comprises oxygen-coordinated NBC. In one aspect, it is contemplated that the composition used in these methods comprises NBC, D-ribose, withanolides, phenylalanine, caffeine, taurine and glutamine.

It is contemplated that the invention provides a method for increasing ATP levels in a person or other mammal comprising: identifying a person or other mammal that can benefit from increased ATP levels; and administering to the person or other mammal a composition comprising chromium, D-ribose, withanolides, phenylalanine, caffeine, taurine and glutamine in an amount sufficient to increase ATP level in the person or other mammal.

Creatine is a nitrogenous organic acid found in muscle tissue of vertebrates mainly in the form of creatine phosphate (CP). The formation of CP is catalyzed by creatine kinase. CP is capable of storing and providing energy for muscular contraction. CP can also act as a marker for the ability to increase muscle mass in the body. In a related embodiment, the invention contemplates a method for increasing CP levels in a person or other mammal comprising: identifying a person or other mammal that can benefit from increased CP levels; and administering to the person or other mammal a composition comprising chromium, D-ribose, withanolides, phenylalanine, caffeine, taurine and glutamine in an amount sufficient to increase CP levels in the person or other mammal.

Serotonin is a monoamine neurotransmitter synthesized from the amino acid tryptophan by enterochromaffin cells in the stomach and bronchi. Serotonin is distributed within the central nervous system and is involved in the regulation of mood. Serotonin imbalances are implicated in certain mental disorders, such as depression and anxiety. Patients with carcinoid syndrome will have high levels of serotonin in blood and urine. The invention also provides a method for increasing serotonin levels in a person or other mammal comprising: identifying a person or other mammal that can benefit from increased serotonin levels; and administering to the person or other mammal a composition comprising chromium, D-ribose, withanolides, and at least one amine, in an amount sufficient to increase serotonin levels in the person or other mammal.

Dopamine is also a monoamine neurotransmitter formed in the brain by the decarboxylation of 3,4-dihydroxy-L-phenylalanine or dopa, which is in turn formed in the liver from the amino acid tyrosine through the action of the enzyme tyrosine hydroxylase. Dopamine acts in the brain and regulates movement, balance, and walking. Dopamine is also linked to sexual desire and pleasure. A dopamine deficiency in certain areas of the brain is associated with Parkinson's disease. In a related aspect the invention provides a method for increasing dopamine levels in a person or other mammal comprising: identifying a person or other mammal that can benefit from increased dopamine levels; and administering to the person or other mammal a composition comprising chromium, D-ribose, withanolides, and at least one amine, in an amount sufficient to increase dopamine levels in the person or other mammal.

Heat shock protein 70 (HSP70) and heat shock protein 80 (HSP80) are member of a class of “chaperone” proteins which assist in the folding of other newly synthesized proteins. In addition to binding hydrophobic regions of assisted proteins, HSP70 and HSP80 are able to bind adenosine triphosphate. HSP70 and HSP80 also play important roles in the transport of proteins across membranes. Both HSP70 and HSP80 exhibit heat shock activity. In a further aspect, the invention contemplates a method for increasing HSP70 levels in a person or other mammal comprising: identifying a person or other mammal that can benefit from increased HSP70 levels; and administering to the person or other mammal a composition comprising chromium, D-ribose, withanolides, and at least one amine, in an amount sufficient to increase HSP70 levels in the person or other mammal.

In a still further aspect, the invention provides a method for increasing HSP80 levels in a person or other mammal comprising: identifying a person or other mammal that can benefit from increased HSP80 levels; and administering to the person or other mammal a composition comprising chromium, D-ribose, withanolides, and at least one amine, in an amount sufficient to increase HSP80 levels in the person or other mammal.

In another aspect, the invention contemplates a method for increasing ubiquitin gene expression in a person or other mammal comprising: identifying a person or other mammal that can benefit from increased ubiquitin gene expression; and administering to the person or other mammal a composition comprising chromium, D-ribose, withanolides, and at least one amine, in an amount sufficient to increase ubiquitin gene expression in the person or other mammal.

It is contemplated that when the invention provides a method for increasing serotonin levels, a method for increasing dopamine levels, a method increasing heat shock protein 70 (HSP70) or HSP80 levels, or a method for increasing ubiquitin gene expression, the composition of the method comprises at least one amine selected from the group consisting of phenylalanine, caffeine, taurine and glutamine. It is further contemplated that the chromium comprises NBC. In one embodiment, the chromium comprises oxygen-coordinated NBC. In one aspect, it is contemplated that the composition used in these methods comprises NBC, D-ribose, withanolides, phenylalanine, caffeine, taurine and glutamine.

### DETAILED DESCRIPTION OF THE INVENTION

The present invention provides compositions and methods for enhancing energy levels and reducing the detrimental effects of stress in an individual. The method includes administering compositions comprising chromium and other natural extract/components to provide enhanced energy levels and reducing the effects of stress in an individual. The composition contemplated by the invention comprises chromium, D-ribose, extracts of Ashwagandha root, and selected amines.

As used herein “enhancing energy levels” or “enhanced energy levels” means, as a subjective measure, wherein the individual receiving the composition has feelings of increased energy and activity compared to before administration of the composition of the invention. “Enhancing energy levels” may also mean to increase biological levels of ATP and creatine phosphate (CP) when compared to levels of these molecules before administration of the composition.

As used herein “reducing stress effects” or “reducing the effects of stress” means to reduce the feelings or conditions in an individual typically associated with stress, including, but not limited to, fatigue, anxiety, malaise, and immunosuppression. “Reducing stress effects” also means that biological activity of stress related proteins, including, but not limited to, heat shock proteins such as HSP70 and HSP80, is modulated so as to improve the physiological response to a stress. The stress described herein may be either environmental stress or stress related to a physiological condition, e.g., oxidative stress.

As used herein, “increasing” when used with respect to increasing serotonin levels or dopamine levels, and the like, means to raise the levels of the molecule being increased to levels above levels of the molecule observed prior to administration of the composition of the invention in a statistically significant average of tests undertaken.

As used herein, an “amount sufficient” refers to an amount of the composition of the invention effective to alleviate the symptoms.

The composition of the present invention comprises the combination of chromium, D-ribose, withanolides, and at least one amine, wherein the amine is selected from the group consisting of phenylalanine, glutamine, caffeine and taurine, for use as a nutraceutical energy formulation. It is understood by a worker of ordinary skill in the art that the formulation of these components may be varied to confer a therapeutic effect to an individual or for a specific condition being treated. For example, if the number of amines included in the composition may be varied, then the percent composition of each ingredient may be different among formulations, depending on the number of ingredients in the composition. A person of ordinary skill will readily be able to determine the composition of the formulation and assess the therapeutic efficacy of each formulation using standard techniques in the art. The composition of the nutraceutical energy formulation may include, but is not limited to, the formulation exemplified in Example 1.

Chromium (Cr) is an essential trace element required for normal protein, fat and carbohydrate metabolism. Chromium levels are known to decrease with age, and marginal chromium deficiencies appear to be widespread. Chromium is necessary for insulin function as it is the biologically active component of glucose tolerance factor (GTF). Chromium extracted from Brewers yeast, which is in the GTF form, is absorbed better than inorganic chromium. GTF is transported across the placental barrier, has different tissue distribution from that of inorganic chromium, and has access to the body pool of chromium that responds to increases in blood insulin. The biologically active form of chromium (GTF) is an essential dietary agent that potentiates the action of insulin and functions in regulating protein, fat and carbohydrate metabolism, thereby regulating energy production.

The GTF-Cr formula contemplated for use is a novel NBC composition. NBC compositions such as those described herein are also known as chromium-nicotinate GTF material, polynicotinate and are preferably available commercially as ChromeMate® from InterHealth Nutraceuticals, Inc., Benicia, Calif. The ChromeMate® brand of NBC is made from at least an alkali metal salt of nicotinic acid, a trivalent chromium salt and adjuvants. The composition and process for synthesizing the ChromeMate® brand of NBC is disclosed in U.S. Pat. Nos. 5,194,615, 4,954,492 and 4,923,855, incorporated herein by reference. In general, a dissociable form of alkali metal, like an alkali metal hydroxide and nicotinic acid are combined to form the alkali metal salt of nicotinic acid. This salt is then reacted with trivalent chromium salt to yield the chromium-nicotinate GTF material (NBC). The ChromeMate® brand of NBC can also be synthesized by reacting the alkali metal salt of nicotinic acid, obtained from other sources as known in the art, with the chromium salt. These reactions can be made with minimum temperature controls, from about 5° C. to about 60° C., and are usually performed in a polar solvent system, like an aqueous or alcohol solvent. The crude chromium nicotinate can be washed, in water or other solvent in which the ChromeMate® NBC is insoluble, to remove any soluble compounds or dried at about 10° C. to about 150° C. at less than 100% relative humidity. ChromeMate® brand of NBC is believed to be comprised of at least a substantial portion of a tri-nicotinic chromium complex, where 1 mg of ChromeMate® NBC contains 100 μg of elemental chromium. A representative structure of ChromeMate® brand NBC is shown in U.S. Pat. No. 5,194,615.

D-Ribose is a five-carbon sugar found in all living cells. D-ribose provides the backbone to several essential molecules, including DNA and ATP. Ribose is used to make ATP, and adding ribose to ones diet should increase the rate at which ATP is generating in vivo, adding to increased energy. Ingestion of ribose bypasses the slow conversion process to make ATP, and ribose is readily incorporated into the adenine di-phosphate (ADP) or mono-phosphate (AMP) forms to more quickly re-create ATP. Ribose has also been attributed as having anti-depressant factors.

Withanolides are the active ingredient in Ashwaghanda (Withania) roots, such as Withania somnifera. Withanolides are steroid lactones that have been shown to have anti-inflammatory and anti-arthritic activity, and show significant anti-cancer activity [Devi et al., Int. J Radiol. Biol. (1996) 69: 193-7].

The select amines described above may be selected from the group consisting of phenylalanine, glutamine, caffeine and taurine. Phenylalanine and glutamine are essential amino acids, which play an important role in general health and nutrition. Caffeine, a purine derivative, has been associated with increasing energy levels through modulation of Ca²⁺ release. Taurine is a sulfur-containing amino acid that has been shown to decrease during aging and is believed to be important for cytoprotection. It has been found in high concentrations in many mammalian excitable cells and is reported to have a variety of functions including osmoregulation, modulation of neuronal excitability, antioxidation and control of Ca²⁺ homeostasis [Huxtable, Physiol. Rev. (1992) 72: 101-63]. Taurine has been shown to be an effective scavenger of hypochlorous acid (HOCI), which is associated with tissue damage in inflammation and injury.

It is contemplated that in addition to its broad affects on energy levels and reduction of stress effects, the compositions of the invention may regulate specific molecules involved in energy, metabolism, mood elevation, and cardiac health.

Serotonin (5-HT), a vital neurotransmitter, is involved in a wide range of behavioral functions in the body, including mood, sleep, and eating behavior. Serotonin deficiency has been implicated in a variety of conditions, including depression, low energy, anxiety, affective disorder, obsessive-compulsive behavior, overeating, insomnia, schizophrenia, migraine headaches and bulimia. Increased plasma levels of serotonin are associated with increased energy expenditure.

Methods for increasing serotonin levels in persons suffering from serotonin deficiency have included use of serotonin selective re-uptake inhibitors, (e.g., fluoxetine), compounds promoting production of serotonin, (e.g., St. John's Wort), or compounds inhibiting the degradation of serotonin. (e.g., monoamine oxidase inhibitor antidepressants). These products, while somewhat effective, do not provide ideal results in all cases, and they also may result in negative side effects in persons ingesting them. Serotonin is a marker for assessing mood elevation in an individual.

Dopamine is a neurotransmitter in the brain. Dopamine is a member of the catecholamine family, and is a precursor to epinephrine (adrenaline) and norepinephrine (noradrenaline). Dopamine is synthesized in the body by decarboxylation from L-DOPA by aromatic-L-amino-acid decarboxylase. Dopamine can be supplied as a drug, which acts on the sympathetic nervous system, producing effects such as increased heart rate and blood pressure. Dopamine is a marker for assessing mood elevation in an individual.

Heat shock proteins (HSPs), especially HSP70 and HSP80, are expressed when cells are exposed to various types of stress and may provide protection against cellular insult. Studies have demonstrated that heat-shock protein 70 (Hsp70), which is induced by environmental stress, acts as a protective mechanism to regulate the damaging effects of environmental or oxidative stress. Heat shock proteins are useful markers for assessing the presence biological response to stress.

Ubiquitin (Ub) has been shown to be upregulated in ischemic brain and heart, indicating it likely plays a role in repair after injury. Ubiquitin is a marker for cardiac health. In accordance with the present invention, the method and composition of the present invention can further include any inert ingredients or diluents, such as sugars or fillers, commonly used in food and drug related products or presently known in the art. In accordance with the present invention, the compositions described herein can be provided in any form presently known to those skilled in the art, including, but not limited to, dietary supplements pill, tablet, capsule, powder, lozenge, gum, or liquid. The step of administering the composition includes administering the composition as part of foods or beverages, including, but not limited to, bars, shakes, drinks, and other processed or prepared foods or beverages. As one skilled in the art would appreciate, the energy formulation may also be administered by injection if appropriate. Actual methods for preparing parenterally administrable compositions will be known or apparent to those skilled in the art and are described in more detail in, for example, Remington's Pharmaceutical Science, 15th ed., Mack Publishing Company, Easton, Pa. (1980).

The actual amount of the composition administered to a person as part of the method and composition of the present invention varies depending upon various factors, including, but not limited to, the person's age, physical condition and body mass. In light of these and other factors that could affect the efficacy of the composition and method, the present invention includes administration of an amount of specific composition that enhances energy levels and/or reduces stress levels in an individual.

Additional aspects and details of the invention will be apparent from the following examples.

### EXAMPLE

Many compositions have been produced to address the nutritional deficits causing such ailments as decreased energy and detrimental effects of stress on the body. However, many therapies merely address one of these conditions individually and do not take into account the effects of energy on stress, and vice versa. To address the problems, a novel nutraceutical energy formulation (NEF), comprising NBC, D-ribose, Ashwagandha extract and selected amines (phenylalanine, glutamine, caffeine, and taurine), was administered to test animals and its effects on energy and stress factors was assessed.

Male Sprague-Dawley rats (18 male and 18 female rats) were fed 25 ppm NEF [0.4% NBC (containing 10% chromium), 10.72% Ashwagandha (containing 5% withanolides), 10.72% D-ribose, 10.72% Phenylalanine, 22.78% caffeine, 22.33% taurine, 22.33% glutamine] for 90 consecutive days. Body weight, feed and water intake, and routine physical activities were regularly monitored. Hearts were isolated at 30, 60 and 90 days post treatment. The animals were sacrificed by CO₂ asphyxiation, approximately 1 min in 95% CO₂ chamber, and the heart quickly removed using common surgical techniques. The heart was placed on dry ice and stored at −80° C. until the assays were carried out. The efficacy of NEF was evaluated by examining the myocardium for the levels of myocardial high-energy phosphate compounds, such as, ATP and CP, using high-pressure liquid chromatography. ATP and CP were separated at 210 nm wavelength. The retention times of CP and ATP were 5.7 and 28.1 min, respectively, making a total run time of 35 min. The homogeneity of the CP and ATP peaks in the rat myocardium extracts were confirmed by the retention times of standards, chromatography of spiked samples, acid degradation of CP, enzymatic degradation of peaks, and the absorbance ratios at various wavelengths. HPLC analysis indicated that myocardial ATP levels in NEF treated animals increased by 7%, 58% and 59% at the 30, 60 and 90 day time points, respectively, compared to the control group.

Expression of stress related proteins; such as heat shock protein 70 (HSP70) were determined by Western blot analysis. HSP70 levels were increased by approximately 20% at both the 30-day and 60 day time points. However, at 90 days, the HSP70 protein levels in NEF treated animals were comparable to those in the vehicle control group.

These results demonstrate that NEF activates HSP70 and preserved ATP and CP levels in the heart, which are considered important energy factors for the proper functioning of the heart.

It is expected that the NEF composition will up regulate serotonin and dopamine in a subject receiving the composition, thereby elevating the individual's mood and general feeling of well being. It is expected that the NEF composition will up regulate ubiqutin gene expression in cardiac cells, indicating an increased rate of repair of these cells. This increased repair contributes to the overall cardiac health of the subject receiving the NEF composition.

Numerous modifications and variations in the invention as set forth in the above illustrative examples are expected to occur to those skilled in the art. Consequently, only such limitations as appear in the appended claims should be placed on the invention.

What is claimed is:

1. A composition for enhancing energy levels and/or decreasing stress in a mammal, comprising chromium, D-ribose, a withanolide and at least one amine.

2. The composition of claim 1, wherein the at least one amine is selected from the group consisting of phenylalanine, caffeine, taurine and glutamine.

3. The composition of claim 1, wherein the chromium comprises niacin-bound chromium (NBC).

4. The composition of claim 3, wherein the chromium comprises oxygen-coordinated NBC.

5. The composition of claim 1, wherein the composition is in the form of a pill, tablet, capsule, lozenge, gum, liquid, powder, food, beverage or other orally administered form.

6. A method for enhancing energy levels and/or decreasing stress in a person or other mammal, comprising administering to the person or other mammal the composition comprising chromium, D-ribose, a withanolide and at least one amine.

7. The method of claim 6, wherein the at least one amine in the composition is selected from the group consisting of phenylalanine, caffeine, taurine and glutamine.

8. The method of claim 6, wherein the chromium comprises NBC.

9. The method of claim 6, wherein the chromium comprises oxygen-coordinated NBC.

10. The method of claim 6, wherein said composition is sufficient for promoting mood elevation in a person or other mammal.

11. The method of claim 6, wherein said composition is sufficient for improving cardiovascular health in a person or other mammal.

12. The method of claim 7, wherein said composition is sufficient for promoting mood elevation in a person or other mammal.

13. The method of claim 7, wherein said composition is sufficient for improving cardiovascular health in a person or other mammal.

14. The method of claim 8, wherein said composition is sufficient for promoting mood elevation in a person or other mammal.

15. The method of claim 8, wherein said composition is sufficient for improving cardiovascular health in a person or other mammal.

16. The method of claim 9, wherein said composition is sufficient for promoting mood elevation in a person or other mammal.

17. The method of claim 9, wherein said composition is sufficient for improving cardiovascular health in a person or other mammal.

18. The method of claim 6, wherein said composition is sufficient for increasing serotonin levels in a person or other mammal.

19. The method of claim 6, wherein said composition is sufficient for decreasing ubiquitin levels in a person or other mammal.

20. The method of claim 6, wherein said composition is sufficient for reducing heat shock proteins in a person or other mammal.

21. A method for enhancing energy levels in a mammal or in a person, comprising administering to the person or other mammal the composition comprising chromium, D-ribose, a withanolide, and at least one amine.

22. A method for decreasing stress levels in a mammal or in a person, comprising administering to the person or other mammal the composition comprising chromium, D-ribose, a withanolide, and at least one amine.

23. The method of claim 18, wherein the chromium comprises NBC.

24. The method of claim 19, wherein the chromium comprises NBC.
